(19) United States
(12) Patent Application Publication
(10) Pub. No.: US 2003/0215441 A1
(43) Pub. Date: Nov. 20, 2003

(54) Process for preparing an antimicrobial composition

(75) Inventor: Bertrand Letartre, Lille-Hellemmes (FR)

Correspondence Address: John S. Egbert, Harrison & Egbert, 412 Main Street, 7th Floor, Houston, TX, 77002, US

(73) Assignee: LABORATORIES ANIOS, Societe Anonyme, Lille-Hellemmes (FR)

(21) Appl. No.: 10/463,308

(22) Filed: Jun. 18, 2003

Related U.S. Application Data
(63) Continuation-in-part of application No. 09/673,580, filed on Dec. 1, 2000, now abandoned.
Application No. 09/673,580 is a 371 of international application No. PCT/FR00/00523, filed on Mar. 2, 2000.

(30) Foreign Application Priority Data
Mar. 5, 1999 (FR) 99/02927

(51) Int. Cl. A61K 31/55; A61K 33/40

(52) U.S. Cl. 424/126; 424/616; 514/212.03

(57) ABSTRACT

A process for preparing an anti-microbial composition including mixing a liquid oxygen-generating base with a liquid acetyl radical generator. The generator contains n-acetylcaprolactam of between 40% and 60% inclusive by weight, alcohol of between 1% and 10% inclusive by weight, a surface active agent of between 0.001% and 20% inclusive by weight, a coloring agent of between 0.001% and 0.01% inclusive by weight, an anti-corrosive agent of between 0.001% and 40% inclusive by weight, and a glycolic derivative of between 0.001% and 10% inclusive by weight.

### RELATED U.S. APPLICATIONS

The present application is a continuation-in-part of U.S. patent application Ser. No. 09/673,580, filed on Dec. 1, 2000, and entitled “Method for Preparing an Antimicrobial Composition”, currently pending, based upon a pending petition to revive to establish copendency. U.S. patent application Ser. No. 09/673,580 claimed priority in French Application No. 99/02.927, filed on Mar. 5, 1999, through International Application No. PCT/FR00/00523, filed on Mar. 2, 2000.

### STATEMENT REGARDING FEDERALLY SPONSORED RESEARCH OR DEVELOPMENT

Not applicable.

### REFERENCE TO MICROFICHE APPENDIX

Not applicable.

### FIELD OF THE INVENTION

The present invention relates to processes for preparing antimicrobial compositions. The present invention also relates to groups of solutions for implementing such a process. The present invention also relates to the use of the antimicrobial composition for disinfecting objects and/or surfaces, for example surgical instruments, endoscopes or circuits.

### BACKGROUND OF THE INVENTION

At present, when it is desired to use objects that are potentially microbially contaminated, it is known to treat them with the aid of disinfecting compositions. To date, use is made of solutions containing, among others, aldehydes and, in particular, formic, glyoxalic, glutaric, succinic, or even orthophthalic aldehyde. These molecules are known for their antimicrobial efficiency. One common practice, used for treating instruments, is to place these solutions in partially closed tanks in which the objects to be treated are soaked.

Unfortunately, these products have the physico-chemical characteristic of being extremely volatile. A release of aldehyde vapors gives rise to verity of drawbacks and difficulties. Various procedures are adopted to accommodate such volatile products.

Other proposed solutions take the form of powders that must be solubilized prior to use. This has the attendant risk, in particular, that a certain non-solubilized amount of this powder can be drawn into circuits, tubes and other passageways and thereby clog them, at least partially.

Other molecules or associations of molecules have also been used, such as chlorine dioxide, hydrogen peroxide, or mixtures of hydrogen peroxide, peracetic acid and acetic acid. Unfortunately, these do not provide a solution to the problem of vapor release from the soaking baths. They also fail to ensure adequate efficacy within an acceptable contact time. As a result, such compositions are not entirely satisfactory when they are used intensively in medical, hospital, industrial, domestic or other environments.

Various patent have issued in the past which describe various types of antimicrobial solutions. For example, U.S. Pat. No. 3,684,477, issued on Aug. 15, 1972 to Blumbergs et al., discloses the addition of hydrogen peroxide, a compound able to release hydrogen peroxide in an aqueous solution, and an organic compound comprising acetyl substituents. This organic compound can be selected from among four families of molecules. In particular, the compound supplying hydrogen peroxide is sodium perborate tetrahydrate. It is preferable to use this salt of sodium because it also makes it possible to maintain an alkaline pH. This patent also provides various examples which implement sodium perborate tetrahydrate as a supplier of hydrogen peroxide in the aqueous solution and various molecules containing acetyl radicals.

International Application No. PCT/GB94/00229, published on Aug. 18, 1994 as International Publication WO 94/18298, teaches a process during which a molecule, that is a source of peroxygen, reacts with an activator compound of the N-acyl type having more than two carbon atoms, in an acidic condition. The source of peroxygen can be hydrogen peroxide or a salt of the perborate or percarbonate type. The reactions take place in an acidic condition, or even very acidic condition. This publication indicates that good results are obtained for a pH less than 4. The results relate to the brilliance of the stained fabric before washing compared to the brilliance after washing. The activators used are ethylenediamine tetraacetic acid or 1,5-diacetyl-2,4-dioxohexahydro-1,3,5-triazine. These activators come from chemical families separate from that of the lactams. The products can be made in the solid or liquid form. In solid form, the products are a powder, obtained by extrusion, pulverization, and drying or granulation. If the product is in the liquid form, the particles of the activator must be put in suspension in the liquid, while protecting them using an encapsulation.

U.S. Pat. No. 5,077,008, issued on Dec. 31, 1991 to Kralovic et al., teaches an antimicrobial composition comprising a strong oxidant and various additives, including anti-corrosion additives and moistening agents. The oxidants used are, i.e., the hydrogen peroxide compounds supplying active oxygen, are compounds supplying chlorine, and or other molecules that are strongly oxidant. When peracetic acid is used, it is also necessary to use a stabilizer in order to increase the duration of the life of the product. A buffer is used at a neutral pH.

U.S. Pat. No. 5,458,802, issued on Oct. 17, 1995 to Sanderson et al., describes a liquid composition containing a lye with a perborate basis having stability improved by putting it in suspension in a non-aqueous liquid. This liquid is a salt of superperborate. This makes it possible to avoid pulverization-drying for the production of a powder product. As a result, enormous amounts of energy are avoided. The alkaline metal superperborate is used in suspension in a non-aqueous liquid that comprises at least one surfactant. An activator is used which reacts with hydrogen peroxide in order to form a peroxidized organic space. Several families of activators are proposed. The liquid suspension is at a pH between 8.5 and 10 when the activator couple of alkaline superborate is used in order to improve the perhydrolysis reaction.

British Patent No. 1,596,313 teaches a process for textile cleaning. In this patent, a lye is provided that does not contain perborate. The use of perborate as a source of hydrogen is indicated as providing numerous problems of efficacy for temperatures above 60° C.

It is an object of the invention is to provide a process for preparing an antimicrobial composition which removes the difficulties associated with the solubilizing powder and to avoid the risks of deposits forming on surfaces and within the circuits and tubes.

It is another object of the invention is to provide a process for preparing an antimicrobial composition which reduces the release of vapors of active components.

It is another object of the invention is to provide a process for preparing an antimicrobial composition which reduces the release of irritating vapors from the individual ingredients.

It is another object of the invention is to provide a process for preparing an antimicrobial composition which provides stability of the solutions over a long period of time under standard storage conditions.

It is still a further object of the invention is to provide an antimicrobial composition which reduces the contact time needed to obtain an effective treatment, in particular for sporulated forms of bacteria.

It is still a further object of the invention is to provide a process for preparing an antimicrobial composition which is very efficient as a disinfectant.

It is another object of the invention is to provide a process for preparing an antimicrobial composition that optimally reduces the reactivity of its components with ferrous and non-ferrous metals.

It is another object of the invention is to provide an antimicrobial composition which has a pH that is as close as possible to neutral.

These and other objects and advantages of the present invention will become apparent from a reading of the attached specification and appended claims.

### BRIEF SUMMARY OF THE INVENTION

The present invention is a process for preparing an antimicrobial composition comprising mixing a liquid oxygen-generating base with a liquid acetyl radical generator. The generator is constituted at least by n-acetylcaprolectem of between 40% and 60% inclusive by weight, alcohol of between 1% and 10% inclusive by weight, a surface active agent of between 0.001% and 20% inclusive by weight, a coloring agent of between 0.001% and 0.01% inclusive by weight, an anti-corrosive agent of between 0.001% and 40% inclusive by weight and a glycolic derivative of between 0.001% and 10% inclusive by weight in which a total amount of components of said generator is no more than 100%. The step of mixing step is carried out extemporaneously.

In the process of the present invention, peracetic acid is produced through a perhydrolysis reaction subsequent to said step of mixing. The process further includes formulating the base from at least stabilized hydrogen peroxide. In the preferred form of the present invention, the base has at least 0.25% hydrogen peroxide. In particular, the base is constituted by hydrogen peroxide of between 0.⁵% and 35% inclusive by weight, a stabilizer of between 0.001% and 5% inclusive by weight, an anti-corrosive agent of between 0.001% and 2% inclusive by weight, a pH regulator of between 0.001% and 5% inclusive by weight, and water in amount sufficient such that the total components of the base is 100%.

In the process of present invention the base is first placed in a first container and the generator is placed in a second container. The steps of placing the base and the generator are carried out prior to the step of mixing.

In the preferred embodiment of the present invention, the n-acetycaprolactam is in an amount of approximately 50% by weight and the alcohol is in an amount of approximately 5% by weight.

### DETAILED DESCRIPTION OF THE INVENTION

In the present invention, surface-active agents, capable of reinforcing antimicrobial activity and/or the solubility of the generator, anti-corrosion agents, pH regulating agents and/or other solvents of the alcohol and/or glycol-type are provided in the base and/or in the generator. These can be added, as required, to the composition before or after mixing the base in the generator. The base is constituted by liquid oxidizing agents, such as hydrogen peroxide. The base is formulated, in particular, from at least stabilized hydrogen peroxide having a given pH. The base is concentrated according to its readiness for use after the addition of the generator. The base can also be diluted with water of suitable quality before use or before addition of the generator. The quantity of hydrogen peroxide is capable of reinforcing antimicrobial activity and for causing the synthesis of the parasitic acid after mixing the base and the generator. Preferably, the quantity of the hydrogen peroxide is at least 0.25% by weight.

The base further contains pH stabilizing and/or regulating agents in order to stabilize the hydrogen peroxide during storage. These pH stabilizing and/or regulating agents can stabilize the mixture over a period of time and can intervene in the kinetics of the perhydraulis reaction when the base and the generator are mixed. The pH of the base can be adjusted, in particular, in order to produce the desired quantity of peracidic acid or of other antimicrobial compounds within as short a period of time as possible. These pH stabilizing and/or regulating agents are constituted by alkaline compounds, such as sodium hydroxide, potassium hydroxide and the like. They can also be, in particular, acids of mineral or organic types and can have a buffer effect, if necessary. These pH stabilizing and/or regulating agents can also be of the monoethanolamine, diethanolamine, triethanolamine and similar types.

The base also contains, as required, anti-corrosion agents. These anti-corrosion agents have the ability to reduce the different forms of corrosion of ferrous and non-ferrous metals. These anti-corrosion agents can be phosphates, phosphonates, triazole derivatives and the like. The base can also contain a coloring reagent or indicator that reacts by changing color. It is desired that the color change when the generator is added.

The generator is constituted by n-acetylcaprolactam. N-acetylcaprolactam permits the release of oxidizable acetyl radicals at the time of mixing. In the case of peracetic acid production, the quantity of generator of the acid added to the base determines the proportion of peracetic acid produced when the base and the generator are mixed.

Different families of components may, as required, be incorporated in the generator in order to optimize a number of aspects. For example, surface active agents may be incorporated so as to reinforce antimicrobial activity and to ensure better penetration of hollow bodies, cracks and crevices. Such a product can also improve dissolution of the acetyl radical generating compound used, such as, in particular, N-acetylcaprolactam. The surface active agents are of a non-ionic type, such as ethylene or propylene oxide condensates, alkyl polyglucosides or ethoxylated and propoxylated fatty acids. The surface active agents can also be of the anionic type such as sulphates, sulphonates or sulphosuccinates. The surface active agents can also be of the amphoteric or cationic type.

Compounds can be included in the generator which enhance the dispersion and/or solubility of the generator in the base. These compounds can be, for example, aliphatic or cyclic alcohols, such as ethanol, isopropanol, n-propanol, butanol. Additionally, the compounds can also be glycolic derivatives, such as monoethyleneglycol, monopropyleneglycol, butylglycol and/or others. The generator can further contain coloring agents or colored indicators which react when the generator is added to the base.

The proportions, by weight, of the various components of the base and of the generator are provided in a table herein below:

TABLE I
Base:

| | |
|---|---|
| Hydrogen peroxide: | 0.5%-35%, in particular 2 to 4% |
| Stabilizers: | 0.0001%-5%, in particular 0.0005 to 0.009% |
| Anti-corrosion agents: | 0.001%-2%, in particular 0.04 to 0.06% |
| pH regulator: | 0.001 to 5%, in particular 0.01 to 1% |
| Water: | quantity sufficient for 100% |
| N-acetyleaprolactam: | 1%-80%, in particular 40 to 60% |
| Alcohol: | 5%-80%, in particular 30 to 40% |
| Surface active agents: | 0.001%-20%, in particular 4 to 7% |
| Coloring agent: | suffic. quant., in particular 0.001 to 0.01% |
| Anti-corrosion agents: | 0.001%-40%, in particular 0.001 to 0.01% |
| Glycolic derivative: | 0.001%-10%, In particular 0.5 to 1% |

The pH of the base is fixed at between 6 and 8±0.1.

This proportioning of the ingredients results in the controlled production of peracetic acid, stability of the peracetic acid produced, and antimicrobial efficacy with respect to bacteria yeasts, mold fungi, viruses and bacteria spores. This can be achieved within a short contact time.

Various tests have been conducted which demonstrate the efficacy of the antimicrobial composition produced by the process of the present invention. The following table summarize the results of various microbiological tests designed to test the sporicidal or anti-microbial efficacy of the composition. Table II involves a test in which the activator contains quantities of N-acetylcaprolactam varying between 30 and 60%. Various variables were measured. These variables include the quantity of peracetic acid at the time of mixture, the quantity of peracetic acid after seven days of storage and the time of contact necessary for a reduction of the number of Bacillus cereus equivalent to 5 log.

TABLE II

| Formulation | | | | |
|---|---|---|---|---|
| No. | 1 | 2 | 3 | 4 |
| N- | 60 | 50 | 40 | 30 |
| acetylcaprolactam (%) | | | | |
| Quantity of | 2100 | 1600 | 1100 | 600 |
| peracetic acid | | | | |
| after activation | | | | |
| (ppm) | | | | |
| (indicator | | | | |
| value) | | | | |
| Quantity of | >1400 | 900 | 300 | * |
| peracetic acid | | | | |
| after 7 days | | | | |
| (ppm) | | | | |
| (indicator | | | | |
| value) | | | | |
| Sporicidal | 20 minutes | 30 minutes | 1 hour | ineffective |
| efficacy: time of | | | | |
| contact | | | | |
| necessary for a | | | | |
| reduction of 5 | | | | |
| log on Bacillus | | | | |
| cereus | | | | |

* 400 ppm after 2 days

In evaluating the results of the above TABLE II, the selected solution must meet several criteria. First, it should have good sporicidal efficacy. Secondly, the solution must be compatible with the materials used. Finally, the selected solution must not be toxic.

Formulation No. 1 is not adequate because it has an increased toxicity (higher percentages of peracetic acid in air) and an incompatibility with materials. The criteria of sporicidal efficacy selected is a reduction of 5 log on Bacillus cereus in less than 30 minutes. To the extent that this is the proper criteria, Formulations nos. 3 and 4 are not sufficient. Formulation 2 is the preferred embodiment of the present invention and the preferred concentration of N-acetylcaprolactam in the overall solution.

The following Table III relates to a formulation containing 50% of n-acetylcaprolactam (the preferred embodiment). The percentage of fatty alcohol varies between 1 and 10%. The time in contact with Bacillus subtilis is 20 minutes. The microbial reduction expressed in logarithmic units is measured.

TABLE III

| Formulation | | | | |
|---|---|---|---|---|
| No. | 2A | 2B | 2C | 2D |
| % fatty alcohol | 1% | 2.5% | 4.9% | 10% |
| Contact time | 20 minutes | 20 minutes | 20 minutes | 20 minutes |
| (Bacillus | | | | |
| subtilis) | | | | |
| Microbial | <2 log | 2.2 log | 3.1 log | 3.2 log |
| reduction | | | | |
| Peracetic acid | 600 ppm | | | |
| (ppm) | | | | |

This test confirms that the antimicrobial efficacy of the formulation also depends on the quantity of fatty alcohol that it contains. However, the more the formulation contains fatty alcohol, the more sizeable is its foaming power. As such, it would not be compatible with a use in the endoscopic channels. Additionally, it is observed with this test that there is a threshold of microbial reduction beyond which the increase of the percentage of fatty alcohol does not make it possible to increase approximately the microbial reduction shown in formulations 2C and 2D. As a result, the formulation 2D does not have a very large advantage as far as the microbial reduction is concerned. It has a foaming power that is too great for use in endoscopic channels. As a result, formulation 2C is the preferred concentration of fatty alcohols. This amount is approximately 5%.

In implementing the process of the present invention, the liquid oxygen-generating base is provided in a first dose and the liquid acetyl radical generator is provided in a second dose. The first and second doses are placed in first and second containers, respectively. They are placed in such containers prior to the step of mixing. In order to implement the process for preparing the antimicrobial composition, the contents of the separate containers are mixed. The contents of the container are ready for use and/or pre-proportioned. When the formulation is suitably mixed in reaction, the composition obtained can be used for disinfecting objects and/or surfaces, such as those of surgical instruments, endoscopes or tubing and conduits.

The foregoing disclosure and description of the invention is illustrative and explanatory thereof. Various changes in the details of the described process can be made within the scope of the appended claims without departing from the true spirit of the invention. The present invention should only be limited by the following claims and their legal equivalents.

I claim:

1. A process for preparing an anti-microbial composition comprising:
mixing a liquid oxygen-generating base with a liquid acetyl radical generator, said generator constituted by n-acetylcaprolactam of between 40% and 60% inclusive by weight, alcohol of between 1% and 10% inclusive by weight, a surface active agent of between 0.001% and 20% inclusive by weight, a coloring agent of between 0.001% and 0.01% inclusive by weight, an anti-corrosive agent of between 0.001% and 40% inclusive by weight and a glycolic derivative of between 0.001% and 10% inclusive by weight in which a total amount of components of said generator is no more than 100%.

2. The process of claim 1, said step of mixing being carried out extemporaneously.

3. The process of claim 1, further comprising:
producing peracetic acid through a perhydrolysis reaction subsequent to said step of mixing.

4. The process of claim 1, further comprising:
formulating said base from at least stabilized hydrogen peroxide.

5. The process of claim 1, said base having at least 0.25% hydrogen peroxide.

6. The process of claim 1, said base being constituted by hydrogen peroxide of between 0.5% and 35% inclusive by weight, a stabilizer of between 0.001% and 5% inclusive by weight, an anti-corrosive agent of between 0.001% and 2% inclusive by weight, a pH regulator of between 0.001% and 5% inclusive by weight, and water in amount sufficient such that the total components of said base is 100%.

7. The process of claim 1, said generation being n-acetycaprolactam in an amount of approximately 50% by weight.

8. The process of claim 7, said alcohol being approximately 5% by weight.

9. The process of claim 1, further comprising:
placing said base in a first container; and
placing said generator in a second container, each of said steps of placing being prior to said step of mixing.
